(19) United States
(12) Patent Application Publication
(10) Pub. No.: US 2016/0009898 A1
(43) Pub. Date: Jan. 14, 2016

(54) ISONONYL ESTERS BASED ON FATTY ACIDS OR FATTY ACID MIXTURES FROM TALL OIL OR LINSEED OIL

(71) Applicants: Benjamin Woldt, Bochum (DE); Michael Grass, Haltem am See (DE); Andreas Gevers, Bottrop (DE)

(72) Inventors: Benjamin Woldt, Bochum (DE); Michael Grass, Haltem am See (DE); Andreas Gevers, Bottrop (DE)

(73) Assignee: EVONIK DEGUSSA GMBH, Essen (DE)

(21) Appl. No.: 14/773,649

(22) PCT Filed: Feb. 14, 2014
(86) PCT No.: PCT/EP2014/052910

§ 371 Date: Sep. 8, 2015

(30) Foreign Application Priority Data
Mar. 8, 2013 (DE) 10 2013 203 972.7

(51) Int. Cl. C08K 5/1515; C07D 303/16; C07D 407/06

(52) U.S. Cl. CPC C08K 5/1515; C07D 407/06; C07D 303/16

(57) ABSTRACT

The invention relates to an isononyl ester or isononyl ester mixture of an epoxidized fatty acid or an epoxidized fatty acid mixture, the fatty acid or fatty acid mixture being extracted from tall oil or linseed oil and the average number of epoxide groups per fatty acid being greater than 1.00.

The invention relates to isononyl esters or to an isononyl ester mixture of an epoxidized fatty acid or of an epoxidized fatty acid mixture, the fatty acid or fatty acid mixture having been obtained from tall oil or linseed oil, and the average number of epoxide groups per fatty acid being greater than 1.00. The invention further relates to processes for preparing them, and to their use as plasticizers for polymers.

Patent specification GB 1,020,866 describes epoxidized diesters of fatty acids from tall oil with glycerol, propylene glycol and ethylene glycol.

Patent specification GB 805,252 specifies epoxidized butyl tallate as a plasticizer.

The objectives were on the one hand to provide further esters (or an ester mixture) whose acid fraction originates from fatty acids from naturally occurring oils, and which have good plasticizer properties, and on the other hand to provide a preparation process allowing these esters (or ester mixtures) to be prepared.

The object is achieved by means of an ester/ester mixture according to claim 1.

Isononyl ester or isononyl ester mixture of an epoxidized fatty acid or of an epoxidized fatty acid mixture, the fatty acid or fatty acid mixture having been obtained from tall oil or linseed oil, and the average number of epoxide groups per fatty acid being greater than 1.00.

In one embodiment the oil is tall oil.

In another embodiment the oil is linseed oil.

In a further embodiment the oil is a mixture of different vegetable oils, the fraction of linseed oil or tall oil being greater than 50 per cent by mass, preferably greater than 75 per cent by mass.

In one embodiment the average number of epoxide groups per fatty acid is greater than 1.20, preferably greater than 1.30, very preferably greater than 1.40.

In one embodiment the fraction of saturated fatty acids in the isononyl ester mixture is less than 12 area %, preferably less than 8 area %, more preferably less than 6 area %.

In one embodiment the fraction of saturated fatty acids in the isononyl ester mixture is greater than 1 area %.

As well as the isononyl ester or isononyl ester mixture itself, a process for preparing it is also claimed.

Process for preparing an above-described isononyl ester or isononyl ester mixture, comprising the following process steps:

a1) recovering a fatty acid or fatty acid mixture from tall oil or linseed oil,

b1) epoxidizing the fatty acid or fatty acid mixture,

c1) esterifying the fatty acids or fatty acid mixture with isononanol.

In this process, step c1) may also take place before step b1).

Process for preparing an above-described isononyl ester or isononyl ester mixture, comprising the following process steps:

a2) recovering a fatty acid ester or fatty acid ester mixture from tall oil or linseed oil,

b2) epoxidizing the fatty acid ester or fatty acid ester mixture,

c2) transesterifying the fatty acid ester or fatty acid ester mixture with isononanol.

In this process, step c2) may also take place before step b2).

In one specific embodiment the fatty acid ester described in a2) is a methyl ester of the corresponding fatty acid or fatty acid mixture.

In one preferred process variant the fatty acid methyl ester is first of all prepared and epoxidized. The epoxidized fatty acid methyl ester is subsequently separated into a fraction rich in saturated fatty acid methyl esters and a fraction rich in epoxidized fatty acid methyl esters. This separation may be accomplished by distillation, for example.

Also claimed, furthermore, is the use of an above-described isononyl ester or isononyl ester mixture.

Use of an above-described ester or ester mixture as plasticizer for a polymer selected from the following: polyvinyl chloride, polyvinylidene chloride, polylactic acid, polyurethanes, polyvinylbutyral, polyalkyl methacrylates or copolymers thereof.

Preference here is given to the use of an above-described ester or ester mixture as plasticizer for polyvinyl chloride.

The esters or ester mixtures of the invention may be used as plasticizers for the modification of polymers. These polymers are selected, for example, from the group consisting of: polyvinyl chloride (PVC), polyvinylidene chloride (PVDC), polyacrylates, especially polymethyl methacrylate (PMMA), polyalkyl methacrylate (PAMA), fluoropolymers, especially polyvinylidene fluoride (PVDF), polytetrafluoroethylene (PTFE), polyvinyl acetate (PVAc), polyvinyl alcohol

(PVA), polyvinylacetals, especially polyvinylbutyral (PVB), polystyrene polymers, especially polystyrene (PS), expandable polystyrene (EPS), acrylonitrile-styrene-acrylate (ASA), styrene-acrylonitrile (SAN), acrylonitrile-butadiene-styrene (ABS), styrene-maleic anhydride copolymer (SMA), styrene-methacrylic acid copolymer, polyolefins, especially polyethylene (PE) or polypropylene (PP), thermoplastic polyolefins (TPO), polyethylene-vinyl acetate (EVA), polycarbonates, polyethylene terephthalate (PET), polybutylene terephthalate (PBT), polyoxymethylene (POM), polyamide (PA), polyethylene glycol (PEG), polyurethane (PU), thermoplastic polyurethane (TPU), polysulphides (PSu), biopolymers, especially polylactic acid (PLA), polyhydroxybutyral (PHB), polyhydroxyvaleric acid (PHV), polyesters, starch, cellulose and cellulose derivatives, especially nitrocellulose (NC), ethylcellulose (EC), cellulose acetate (CA), cellulose acetate/butyrate (CAB), rubber or silicones, and also mixtures or copolymers of the stated polymers or of their monomeric units. The polymers of the invention preferably comprise PVC or homopolymers or copolymers based on ethylene, propylene, butadiene, vinyl acetate, glycidyl acrylate, glycidyl methacrylate, methacrylates, ethyl acrylates, butyl acrylates or methacrylates having, bonded on the oxygen atom of the ester group, alkyl radicals of branched or unbranched alcohols having one to ten carbon atoms, styrene, acrylonitrile or cyclic olefins.

The type of PVC in the polymer is preferably suspension PVC, bulk PVC, microsuspension PVC or emulsion PVC.

Based on 100 parts by mass of polymer, the polymers comprise preferably from 5 to 200, more preferably from 10 to 150, parts by mass of plasticizer.

The esters/ester mixtures of the invention may also be combined with other plasticizers, for example with other esters of natural fatty acids, or with oil from plant sources.

Combination may also take place, furthermore, with a plasticizer selected from the following group: adipates, benzoates, citrates, cyclohexanedicarboxylates, epoxidized fatty acid esters, epoxidized vegetable oils, epoxidized acetylated glycerides, furandicarboxylates, phosphates, phthalates, sulphonamides, sulphonates, terephthalates, trimellitates, or oligomeric or polymeric esters based on adipic, succinic or sebacic acid.

The mixtures of PVC and the esters of the invention may also be admixed with other additives as well, such as, for example, heat stabilizers, fillers, pigments, blowing agents, biocides, UV stabilizers, etc.

The above-described esters or ester mixtures may be used in adhesives, sealants, coating materials, varnishes, paints, plastisols, foams, synthetic leathers, floor coverings (e.g. top coat), roofing sheets, underbody protection, fabric coatings, cables or wire insulation systems, hoses, extruded articles, and also in films, particularly for the automotive interior, and also in wallpapers or inks.

Preparation of the Compounds

### EXAMPLE 1

### Isononyl Fatty Acid ester Based on Linseed Oil

Batch:
1320 g linseed oil (from Mosselmann)
1080 g isononanol (from Evonik)
3.30 g tetraisononyl titanate (obtainable by transesterifying tetrabutyl titanate from Johnson Matthey with isononanol from Evonik; nonyl titanate purity 95%)

Transesterification:
All of the reactants and the catalyst were charged to an esterification apparatus with a 4 l distillation flask with stirrer, immersion tube, sampling port, thermometer and water separator mounted with intensive condenser.
The apparatus was flushed via the immersion tube with 6 l N₂/hour for one hour. The reaction as well took place with nitrogen sparging.
The batch was slowly heated to 240° C. with stirring. Under full reflux, the temperature was kept constant. The reaction time was 6 hours.
The conversion was monitored via the decrease in the isononanol fraction, by means of GC analysis. When there was no further change in the isononanol fraction, the batch was shut off and cooled to 80° C.
The reaction effluent from the transesterification was transferred to a 4 L reaction flask, and attached to a Claisen bridge with vacuum divider. Also fitted on were an immersion tube with nitrogen connector, and a thermometer. The batch was flushed with nitrogen while stirring. To remove the glycerol liberated in the reaction, the batch at 80° C. was washed three times with 25% of DI water (based on the amount of reaction effluent) and allowed to settle, after which the aqueous phase was separated off. Subsequently, the excess isononanol still present was distilled off at up to 210° C. (<1 mbar) in the presence of 1% of activated carbon. The vacuum was adjusted to 40 mbar by nitrogen sparging, and the product was cooled to 90° C. When the temperature was reached, the ester was filtered through a Büchner funnel with filter paper and precompacted filter cake of filter aid (D14 perlite), using reduced pressure, into a suction bottle. The filtrate was subjected to determinations of colour number, acid number, water content, density and dynamic viscosity and to a GC analysis.

### EXAMPLE 2

### Epoxidized isononyl Fatty Acid ester Based on Linseed Oil

Batch:
408 g isononyl fatty acid ester from Example 1
430 g 35% strength peracetic acid (Peraclean 35, from Evonik)
102 g DI water
450 ml 20% strength sodium hydroxide solution (from Merck)

The fatty acid ester was charged to an epoxidation apparatus (3000 ml jacketed reactor with integrated cooling coil, stirrer, immersion tube, dropping funnel, metering pump, thermometer, pH meter and mounted reflux condenser). The apparatus was flushed with 61 N₂/hour for 60 minutes via the immersion tube. During the reaction, nitrogen was passed over the reactor contents in order to ensure inertization of the gas phase. The ester was slowly heated to 45° C. with stirring. When the target temperature was reached, the pH was adjusted to 5 using the sodium hydroxide solution. Subsequently, the peracetic acid was added over the course of 2 hours by the metering pump. Over the entire time, the pH was kept constant by dropwise addition of sodium hydroxide solution. The reaction temperature was kept constant (+/−1.5° C.) throughout the reaction time. After the start of the reaction, the heat of reaction was taken off via the cooling coil. Complete addition was followed by subsequent reaction over 22 hours. After reaction times of 10, 30 and 60 minutes, and also 2, 4, 8 and 24 hours, samples were taken and analysed in order to document the reaction course. The sample volume was approximately 3 ml, and was subsequently extracted by shaking with 3 times the amount of DI H₂O. After a settling time of around 30 minutes, the organic phase was separated from the aqueous phase, introduced into an evaporation boat, and dried in a desiccator for 12 hours. The conversion was checked via NMR measurements.

The reaction effluent was introduced into a separating funnel and allowed to settle at room temperature for 30 minutes. The aqueous phase was drained off and discarded. The organic phase was introduced into a 2 litre reaction flask and, with immersion tube, stirrer and thermometer, was fitted to a Claisen bridge with vacuum connection. The reaction effluent was washed 3 times with 25% of water, based on the initial mass. This was followed by drying under maximum vacuum for 15 minutes at 60° C. The contents of the flask were then heated to 160° C. After they had reached this temperature, they were stripped with nitrogen. For this purpose, the amount of nitrogen was set such that the pressure rose from maximum vacuum to 40 mbar.

After 2 hours, the heating was switched off and the contents of the flask were cooled to 90° C. with introduction of nitrogen. The ester was filtered through a Buchner funnel with filter paper and precompacted filter cake of filter aid (D14 perlite), using reduced pressure, into a suction bottle. The filtrate was subjected to a determination of colour number and acid number and also to GC and NMR analyses.

### EXAMPLE 3

### Isononyl Fatty Acid ester Based on Tall Oil Fatty Acids

Batch:
1410 g tall oil fatty acids (UCY TF2, from UCY Energy)
900 g isononanol (from Evonik)
3.53 g tetraisononyl titanate (obtainable by transesterifying tetrabutyl titanate from Johnson Matthey with isononanol from Evonik; nonyl titanate purity 95%)

Esterification:

All of the reactants and the catalyst were charged to an esterification apparatus with a 4 l distillation flask with stirrer, immersion tube, sampling port, thermometer and water separator mounted with intensive condenser.

The apparatus was flushed via the immersion tube with 6 l N2/hour for one hour. The reaction as well took place with metered introduction of nitrogen.

The product was slowly heated with stirring. The onset of boiling was at 172° C. From this time on, water of reaction was obtained, and was removed from the reaction continuously via the water separator. The product was heated further up to the reaction temperature of 240° C. When the reaction temperature was reached, it was kept constant via addition of cyclohexane. In this way the reflux was maintained. In the course of the esterification, 88 ml of water were produced. The reaction time was 6.5 hours.

The conversion was monitored via the amount of water and the acid number. When water of reaction was no longer produced, a sample was taken and its acid number determined. The batch was shut off when the acid number was <0.1 mg KOH/g.

The reaction effluent from the esterification was transferred to a 4 l reaction flask and fitted to a Claisen bridge with vacuum divider. An immersion tube with nitrogen connection, and a thermometer, were also fitted. The batch was flushed with nitrogen while stirring. Under maximum vacuum (<1-5 mbar), heating was carried out slowly, and the temperature was increased to 190° C. slowly in line with the distillation yield. The vacuum was adjusted to 40 mbar via nitrogen sparging, and the product was cooled to 180° C. When the temperature was reached, stripping with nitrogen took place for 2 hours at 180° C. and 40 mbar. The heating was then shut off and the batch was cooled to 100° C. in the stream of nitrogen. The mass and the acid number of the flask contents were determined.

The ester was filtered through a Büchner funnel with filter paper and precompacted filter cake of filter aid (D14 perlite), using vacuum, into a suction bottle. The filtrate was subjected to a colour number, acid number and GC analysis.

### EXAMPLE 4a/b/c

### Epoxidized Isononyl Fatty Acid ester Based on Tall Oil Fatty Acids

Batch:
800 g isononyl tallate (from Example 3)
860 g 35% strength peracetic acid (Peraclean 35, from Evonik)
204 g DI water
690 ml 20% strength sodium hydroxide solution (from Merck)

Epoxidation:

The fatty acid ester was charged to an epoxidation apparatus (3000 ml jacketed reactor with integrated cooling coil, stirrer, immersion tube, dropping funnel, metering pump, thermometer, pH meter and mounted reflux condenser). The apparatus was flushed with 61 N₂/hour for 60 minutes via the immersion tube. During the reaction, nitrogen was passed over the reactor contents in order to ensure inertization of the gas phase. The product was slowly heated to 45° C. with stirring. When the target temperature was reached, the pH was adjusted to 5 using the sodium hydroxide solution. Subsequently, the peracetic acid was added over the course of 2 hours by the metering pump. Over the entire time, the pH was kept constant by dropwise addition of sodium hydroxide solution. The reaction temperature was kept constant (+/−1.5° C.) throughout the reaction time. After the start of the reaction, the heat of reaction was taken off via the cooling coil. Complete addition was followed by subsequent reaction over 22 hours. After reaction times of 10, 30 and 60 minutes, and also 2, 4, 8 and 24 hours, samples were taken and analysed in order to document the reaction course. The sample volume was approximately 3 ml, and was subsequently extracted by shaking with 3 times the amount of DI water. (Example 4a)

Lower degrees of epoxidation were brought about by terminating the reaction after a shorter time (Example 4b 20 hours and Example 4c 4.5 hours).

After a settling time of around 30 minutes, the organic phase was separated from the aqueous phase, introduced into an evaporation boat, and dried in a desiccator for 12 hours. The conversion was checked via NMR measurements.

The reaction effluent was introduced into a separating funnel and allowed to settle at room temperature for 30 minutes. The aqueous phase was drained off and discarded. The organic phase was introduced into a 2 litre reaction flask and, with immersion tube, stirrer and thermometer, was fitted to a Claisen bridge with vacuum connection. The reaction effluent was washed 3 times with 25% of water, based on the initial mass. This was followed by drying under maximum vacuum for 15 minutes at 60° C. The contents of the flask were then heated to 160° C.

After they had reached this temperature, they were stripped with nitrogen. For this purpose, the amount of nitrogen was set such that the pressure rose from maximum vacuum to 40 mbar. After 2 hours, the heating was switched off and the contents of the flask were cooled to 90° C. with introduction of nitrogen. The ester was filtered through a Büchner funnel with filter paper and precompacted filter cake of filter aid (D14 perlite), using reduced pressure, into a suction bottle. The filtrate was subjected to a determination of colour number and acid number and also to GC and NMR analyses.

### EXAMPLE 5

### Isodecyl Fatty Acid ester Based on Tall Oil Fatty Acids

Batch:
1368 g tall oil fatty acids (UCY TF2, from UCY Energy)
613 g isodecanol (Exxal 10 from Exxon)
3.42 g tetrabutyl titanate (Vertec TNBT, from Johnson Matthey Catalysts)

Esterification:

All of the reactants and the catalyst were charged to an esterification apparatus with a 4 l distillation flask with stirrer, immersion tube, sampling port, thermometer and water separator mounted with intensive condenser.

The apparatus was flushed via the immersion tube with 6 l N2/hour for one hour. The reaction as well took place with metered introduction of nitrogen.

The product was slowly heated with stirring. The onset of boiling was at 172° C. From this time on, water of reaction was obtained, and was removed from the reaction continuously via the water separator. The product was heated further up to the reaction temperature of 240° C. When the reaction temperature was reached, it was kept constant via addition of cyclohexane. In this way the reflux was maintained. In the course of the esterification, 88 ml of water were produced. The reaction time was 6.5 hours.

The conversion was monitored via the amount of water and the acid number. When water of reaction was no longer produced, a sample was taken and its acid number determined. The batch was shut off when the acid number was <0.1 mg KOH/g.

The reaction effluent from the esterification was transferred to a 4 l reaction flask and fitted to a Claisen bridge with vacuum divider. An immersion tube with nitrogen connection, and a thermometer, were also fitted. The batch was flushed with nitrogen while stirring. Under maximum vacuum (<1-5 mbar), heating was carried out slowly, and the temperature was increased to 190° C. slowly in line with the distillation yield. The vacuum was adjusted to 40 mbar via nitrogen sparging, and the product was cooled to 180° C. When the temperature was reached, stripping with nitrogen took place for 2 hours at 180° C. and 40 mbar. The heating was then shut off and the batch was cooled to 100° C. in the stream of nitrogen. The mass and the acid number of the flask contents were determined.

The ester was filtered through a Büchner funnel with filter paper and precompacted filter cake of filter aid (D14 perlite), using vacuum, into a suction bottle. The filtrate was subjected to determination of colour number and acid number and also to GC analysis.

### EXAMPLE 6

### Epoxidized isodecyl Fatty Acid ester Based on Tall Oil Fatty Acids

Batch:

422 g isodecyl tallate (from Example 5)

430 g 35% strength peracetic acid (Peraclean 35, from Evonik)

102 g DI water

445 ml 20% strength sodium hydroxide solution (from Merck)

Epoxidation:

The fatty acid ester was charged to an epoxidation apparatus (3000 ml jacketed reactor with integrated cooling coil, stirrer, immersion tube, dropping funnel, metering pump, thermometer, pH meter and mounted reflux condenser). The apparatus was flushed with 61 N₂/hour for 60 minutes via the immersion tube. During the reaction, nitrogen was passed over the reactor contents in order to ensure inertization of the gas phase. The product was slowly heated to 45° C. with stirring. When the target temperature was reached, the pH was adjusted to 5 using the sodium hydroxide solution. Subsequently, the peracetic acid was added over the course of 2 hours by the metering pump. Over the entire time, the pH was kept constant by dropwise addition of sodium hydroxide solution. The reaction temperature was kept constant (+/−1.5° C.) throughout the reaction time. After the start of the reaction, the heat of reaction was taken off via the cooling coil. Complete addition was followed by subsequent reaction over 22 hours. After reaction times of 10, 30 and 60 minutes, and also 2, 4, 8 and 24 hours, samples were taken and analysed in order to document the reaction course. The sample volume was approximately 3 ml, and was subsequently extracted by shaking with 3 times the amount of DI water. After a settling time of around 30 minutes, the organic phase was separated from the aqueous phase, introduced into an evaporation boat, and dried in a desiccator for 12 hours. The conversion was checked via NMR measurements.

The reaction effluent was introduced into a separating funnel and allowed to settle at room temperature for 30 minutes. The aqueous phase was drained off and discarded. The organic phase was introduced into a 2 litre reaction flask and, with immersion tube, stirrer and thermometer, was fitted to a Claisen bridge with vacuum connection. The reaction effluent was washed 3 times with 25% of water, based on the initial mass. This was followed by drying under maximum vacuum for 15 minutes at 60° C. The contents of the flask were then heated to 160° C. After they had reached this temperature, they were stripped with nitrogen. For this purpose, the amount of nitrogen was set such that the pressure rose from maximum vacuum to 40 mbar. After 2 hours, the heating was switched off and the contents of the flask were cooled to 90° C. with introduction of nitrogen. The ester was filtered through a Büchner funnel with filter paper and precompacted filter cake of filter aid (D14 perlite), using reduced pressure, into a suction bottle. The filtrate was subjected to a determination of colour number and acid number and also to GC and NMR analyses.

### EXAMPLE 7

### Isononyl Fatty Acid ester Based on Rapeseed Oil methyl ester

Batch:
1480 g biodiesel from rapeseed oil (from Mosselmann)
900 g isononanol (from Evonik)
3.70 g tetraisononyl titanate (obtainable by transesterifying tetrabutyl titanate from Johnson Matthey with isononanol from Evonik; nonyl titanate purity 95%)

Transesterification:

All of the reactants and the catalyst were charged to a transesterification apparatus with a 4 l reaction flask, stirrer, immersion tube, thermometer, distillation head, 20 cm Raschig ring column, vacuum divider and collecting flask. The apparatus was flushed with 6 l N₂/hour for one hour via the immersion tube.

The reactants were heated slowly to 220° C. with stirring. The onset of boiling was at 164° C. From this point on, methanol was produced, and was removed from the reaction continuously via the distillation head. When 220° C. were reached, vacuum was applied and the pressure was reduced continuously over the course of the reaction. In the course of the transesterification, 130 g of methanol were obtained. The reaction time was 6 hours. At the end of the reaction the vacuum was 300 mbar.

The conversion was checked via GC analysis. The batch was shut off when the fraction of biodiesel was <0.5 area %. The 1^{st} sample was taken after 4.5 hours, and then the conversion was checked by means of GC analyses at regular intervals until the end of reaction.

The reaction effluent from the transesterification is transferred to a 4 l reaction flask and admixed with 2% of activated carbon, based on the mass of reaction effluent. The flask was fitted to a Claisen bridge with vacuum divider. Additionally, an immersion tube with nitrogen connection was inserted into the flask. A thermometer was fitted as well. The batch was flushed with nitrogen, while stirring. Under maximum vacuum (<1 mbar), heating took place slowly and the temperature was raised slowly to 222° C. in line with the distillation yield. The main fraction was taken off in the temperature range from 214° C. to 219° C. The low (<214° C.) and high (>219° C.) boilers were discarded.

### EXAMPLE 8

### Epoxidized isononyl Fatty Acid ester Based on Rapeseed Oil methyl ester

Batch:

200 g isononyl fatty acid ester (Example 7)

21 g formic acid (from Sigma Aldrich)

79 g hydrogen peroxide, 35% strength (from Fluka)

Epoxidation:

The fatty acid ester was charged to an epoxidation apparatus (1000 ml jacketed reactor with integrated cooling coil, stirrer, immersion tube, dropping funnel, thermometer and mounted reflux condenser). The apparatus was flushed with 6 l N₂/hour for 60 minutes via the immersion tube. During the reaction, nitrogen was passed over the reactor contents in order to ensure inertization of the gas phase. The product was slowly heated to 55° C. with stirring. The hydrogen peroxide was subsequently added over the course of 2 hours, using a metering pump. After about 5 minutes, the temperature in the reactor rose. At 60° C., the temperature was held and was kept constant (+/−1.5° C.) by the cooling. After the start of the reaction, the heat of reaction (strongly exothermic reaction) was taken off via the cooling coil (cooling in intervals). Complete addition was followed by subsequent reaction over 5 hours.

The reaction effluent was introduced into a separating funnel and allowed to settle at room temperature for 30 minutes. The aqueous phase was drained off and discarded. The organic phase was introduced into a 0.5 litre reaction flask and, with immersion tube, stirrer and thermometer, was fitted to a Claisen bridge with vacuum connection. This was followed by drying under maximum vacuum for 15 minutes at 60° C. The contents of the flask were then heated to 160° C. After they had reached this temperature, they were stripped with nitrogen. For this purpose, the amount of nitrogen was set such that the pressure rose from maximum vacuum to 40 mbar. After 2 hours, the heating was switched off and the contents of the flask were cooled to 90° C. with introduction of nitrogen. The ester was filtered through a Büchner funnel with filter paper and precompacted filter cake of filter aid (D14 perlite), using reduced pressure, into a suction bottle. The filtrate was subjected to a determination of colour number and acid number and also to GC and NMR analyses.

Comparative experiments for plastisol use

1. Physicochemical data of the pure plasticizer

1.1 Volatility

The volatility of plasticizers is a central property for many polymer applications. High volatilities lead to environmental exposure and, as a result of reduced plasticizer fractions in the polymer, to impaired mechanical properties. For these reasons, volatile plasticizers are often only admixed in small fractions to other plasticizer systems, or are not used at all. The volatility is particularly significant, for example, in interior applications (wallpapers, cars) or, owing to directives and standards, in the case of cables or food packaging. The volatility of the pure plasticizers was determined by means of the Mettler Toledo HB 43-S halogen dryer. Prior to measurement, a clean, empty aluminium boat was placed in the weighing pan. The aluminium boat was then tared with a mat, and about five grams of plasticizer were pipetted onto the mat and weighed accurately.

Measurement commenced with the closing of the heating module, and the sample was heated at maximum rate (preset) from room temperature to 200° C., with the corresponding loss of mass through vaporization being determined automatically by weighing every 30 seconds. After 10 minutes, the measurement was ended automatically by the instrument. A duplicate determination was carried out on each sample.

1.2 Viscosity and Density

The Stabinger SVM 3000 viscometer is a combination instrument which can be used to determine density and viscosity. For this purpose, the instrument has two measuring cells in series.

To determine the viscosity, a rotary viscometer with cylinder geometry is installed, and, to determine the density, a density measuring cell operating on the oscillating U-tube principle. Accordingly, a single injection of the sample provides both measurement values. Sample measurement takes place at 20° C. The measuring cells are conditioned using a Peltier element (reproducibility 0.02° C.).

The samples are measured using the preset measurement mode “M0-ASTM (PRECISE)”, measurement with very high accuracy and repetitions, for tests in accordance with the standard ASTM D7042. For each measurement, about 0.5 ml of sample is metered in (in order to rule out air inclusions or impurities).

For the internal repetitions, a valid result is displayed only when the deviation in the values is not greater than +/−0.1% of the viscosity measurement and +/−0.0002 g/cm3 for the density. In addition to the internal repetitions, a duplicate determination is carried out on each sample. After each determination, the instrument is cleaned with acetone and dried with air (installed pump).

1.3 Description of Method for Determining the Fraction of Double Bonds, Epoxides and Alcohols via NMR Spectroscopy

The fraction of double bonds, epoxides and alcohols is determined by ¹H NMR spectroscopy. For the recording of the spectra, for example, 50 mg of substance are dissolved in 0.6 ml of

CDCI₃ (containing 1% by mass of TMS) and the solution is introduced into a 5 mm diameter NMR tube.

The NMR spectroscopy analyses can be carried out in principle with any commercial NMR instrument. For the present NMR spectroscopy analyses, a Bruker Avance 500 instrument was used. The spectra were recorded at a temperature of 303 K with a delay of d1=5 seconds, 32 scans, a pulse length of about 9.5 μs, and a sweep width of 10 000 Hz, using a 5 mm BBO (broad band observer) sample head. The resonance signals are plotted against the chemical shift from tetramethylsilane (TMS=0 ppm) as internal standard. Comparable results are obtained with other commercial NMR instruments, with the same operating parameters. To determine the fractions of the individual structural elements it is necessary first to identify the associated signals in the NMR spectrum. Listed below are signals used with their position in the spectrum and their assignment to corresponding structural elements:
the signals in the 4.8 to 6.4 ppm region were assigned to the ¹H nuclei of the double bonds.
the signals in the 4.0 to 3.25 ppm region were assigned to the ¹H nuclei of the alcohols.
the signals in the 3.25 to 2.85 ppm region were assigned to the ¹H nuclei of the epoxides.

Quantification of the fractions requires reference signals of known size. Methylene groups of the fatty acid radical or of the alcohol radical of the fatty acid esters were used. In the case of the isononyl and isodecyl esters, signals of the alcohol are partially superimposed on the signal of the methylene group at 2.3 ppm, and therefore the methylene group of the alcohol at around 4 ppm was employed. The signals used were as follows:
the signals of the methylene group adjacent to the carboxyl group of the fatty acid, resonating in the spectrum as a narrow signal multiplet around 2.3 ppm.
the signals of the methylene group adjacent to the oxygen of the esterified alcohol (isononyl alcohol or isodecyl alcohol), corresponding to the structural element —CH₂—O—, which resonate in the spectrum in the 3.9 to 4.2 ppm region.

Quantification takes place by determination of the area under the respective resonance signals, i.e., the area enclosed from the baseline by the signal. Commercial NMR instruments possess devices for integrating the signal area. In the present NMR spectroscopy analysis, the integration was carried out by means of the TOPSPIN software, Version 3.1.

In order to calculate the fraction of the double bonds, the integral value x of the double bond signals in the 4.8 to 6.4 ppm region is divided by the integral value of the reference methylene group r.

To calculate the fraction of the epoxides, the integral value y of the epoxide signals in the 2.85 to 3.25 ppm region is divided by the integral value of the reference methylene group r.

To calculate the fraction of the alcohols, the integral value z of the epoxide signals in the 3.9 to 3.25 ppm region is divided by half the integral value of the reference methylene group r/2.

This gives the relative fractions of the double bond, epoxide and alcohol structural elements for each fatty acid radical.

1.4 Fraction of Saturated Fatty Acids

The fraction of saturated fatty acids in the fatty acid esters was determined by transesterification to the methyl esters, followed by gas chromatography measurements. The samples were worked up in accordance with Ph. Eur. 01/2008:20422 corrected 6.8, Method C (fatty acid determination in polysorbate) and compared with the test mixtures described therein. Sample preparation was carried out as follows:

0.1 g of sample was admixed with 2.0 ml of NaOH solution (20 g NaOH/l anhydrous methanol) and heated under reflux for 30 minutes. Then 2.0 ml of methanolic boron trifluoride solution (140 mg/ml) were added, followed by heating under reflux for a further 30 minutes. Following addition of 4.0 ml of n-heptane, heating under reflux took place for 5 minutes more, after which the reaction solution was cooled to room temperature. The organic phase was extracted once with 10 ml of saturated sodium chloride solution and then a further three times with 2.0 ml of Milli-Q water. The organic phase was dried over about 0.2 g of anhydrous sodium sulphate. The upper, clear phase was used for the analysis.

For the gas chromatography analyses, 2 methods were used, and the results from both measurements were combined.

GC analysis by method 1 took place with the following parameters:

Capillary column: 30 m DB-WAX; 0.32 mm ID; 0.5 μm film

Carrier gas: helium

Total flow rate: about 106 mL/min

Split: about 100 ml/min

Oven temperature: 80° C.-10° C./min-220° C. (40 min)

Injector: 250° C.

Detector (FID): 250° C.

Injection volume: 1.0 μl

The components in the chromatogram of the sample were identified using a comparative solution of the relevant fatty acid methyl esters. In this case the methyl esters in question are those of lauric and myristic, palmitic and stearic acid. This was followed by standardization of the signals in the chromatogram with run times of between 8 and 20 min of the sample to 100 area %. Method 1 permits separation and quantification of the saturated and unsaturated fatty acid methyl esters among one another. For determining the fraction of the saturated fatty acids in the epoxidized fatty acids, the sample (prepared as described above) is diluted 1:10 with heptane and analysed by method 2.

GC analysis by method 2 took place with the following parameters:

Capillary column: 30 m DB-5HT; 0.32 mm ID; 0.1 μm film

Carrier gas: helium

Column flow rate: 2.6 ml/min /

Oven temperature: 80° C.-20° C./min-400° C. (30 min) Injector: cool on column, 80° C.-140° C./min-400° C.

Detector (FID): 400° C.

Injection volume: 1.0 μl

The procedure used for evaluating the area per cent distribution of the saturated fatty acid methyl esters was as follows: first of all, the retention time range of the saturated and unsaturated fatty acid methyl esters was identified using a comparative solution of relevant fatty acid methyl esters. All of the signals of the fatty acid methyl esters (saturated, unsaturated and epoxidized fatty acid methyl esters) as fatty acids were standardized to 100 area %. The fractions of the individual fatty acid methyl esters in area % could then be calculated as follows:

Fraction of the fatty acid methyl esters (saturated and unsaturated by method II in area %) multiplied by the fraction of the respective fatty acid methyl ester (saturated and unsaturated by method I in area %/100%). The fraction of the saturated FA is then given by summing of the fractions of myristic, palmitic and stearic fatty acid methyl ester.

Example PZ No. 2 (Drapex 4.4)

Method 1 supplies the area percentages of the saturated and unsaturated fatty acid methyl esters (epoxidized fatty acid methyl esters are not included):

| | |
|---|---|
| Methyl myristate | 00.00 area % |
| Methyl palmitate | 17.11 area % |
| Methyl stearate | 46.94 area % |
| Remainder | 35.95 area % |
| Total | 100.00 area % |

The sum total of the saturated fatty acids according to method 1 is therefore 64.05 area %.

Method 2 yields the area percentages of the epoxidized fatty acid methyl esters

| | |
|---|---|
| Un/saturated fatty acid methyl esters | 4.85 area % |
| Epoxidized fatty acid methyl esters | 95.15 area % |

The true fraction of saturated fatty acid methyl esters in PZ No. 2 is then calculated as follows: 0.6405 x 0.0485 x 100 area %=3.10691 area %

The results are shown in Table 1. The plasticizer number (PZ No.) here correlates with the formulation number from Table 2.

TABLE 1

| | Loss of mass | | | | | | Fraction of |
|---|---|---|---|---|---|---|---|
| PZ | 200° C./10 min | Viscosity | Density | EN/FA | DB/FA | OHN/FA | sat. FA |
| No. | [%] | [mPas] | [mg/cm³] | [eq.] | [eq.] | [eq.] | [area %] |
| 1 | 4.4 | 76 | 0.9741 | — | — | — | — |
| 2 | 5.4 | 42 | 0.9247 | 1.28 | 0.01 | 0.25 | 3.1 |
| 3* | 2.4 | 54 | 0.9400 | 1.41 | 0.44 | 0.04 | 10.8 |
| 4* | 2.2 | 44 | 0.9270 | 1.42 | 0.24 | 0.05 | 1.5 |
| 5 | 2.2 | 48 | 0.9201 | 1.20 | 0.34 | 0.05 | 1.6 |
| 6* | 2.8 | 48 | 0.9289 | 1.21 | 0.26 | 0.12 | 1.5 |
| 7 | 3.4 | 28 | 0.9065 | 0.74 | 0.86 | 0.10 | 1.5 |
| 8 | 4.0 | 50 | 0.9243 | 1.21 | 0.05 | 0.29 | 17.7 |
| 9 | 2.4 | — | — | 1.13 | 0.04 | 0.02 | 8.2 |
| 10 | 5.5 | — | — | 1.22 | 0.04 | 0.17 | 17.7 |

*inventive ester
EN/FA: average number of epoxide groups per fatty acid
DB/FA: average number of double bonds per fatty acid
OHN/FA: average number of alcohol groups per fatty acid

For the epoxidized 2-ethylhexyl tallate (2) and the epoxidized ethylhexyl soyate (10), the mass losses of the pure plasticizer are above the mass loss for the industry standard DINP (1). High volatilities lead to environmental exposure and, as a result of reduced plasticizer fractions in the polymer, to impaired mechanical properties. The mass losses of the inventive plasticizers (3), (4) and (6) are in each case well below the value measured for DINP (1).

2. Production of the Plastisol

A PVC plastisol was produced, of the type which is used, for example, to fabricate top coat films for floor coverings. The data in the plastisol formulations are in each case in weight fractions.

The PVC used was Vestolit B 7021-Ultra. The comparative substances used were diisononyl phthalate (DINP, VESTINOL 9 from Evonik Industries) and epoxidized 2-ethylhexyl tallate (Drapex 4.4 from Chemtura), an epoxidized 2-ethylhexyl soyate (PLS Green 8 from Petrom), an epoxidized isononyl soyate (PLS Green 9 from Petrom) and an isononyl fatty acid ester based on rapeseed oil fatty acids (Example 8), and also an isodecyl fatty acid ester from tall oil fatty acids (Example 6).

The formulations of the polymer compositions are listed in Table 2.

TABLE 2

| Formulation: | 1 | 2 | 3* | 4* | 5 | 6* | 7 | 8 | 9 | 10 |
|---|---|---|---|---|---|---|---|---|---|---|
| PVC | 100 | 100 | 100 | 100 | 100 | 100 | 100 | 100 | 100 | 100 |
| (B 7021 - Ultra, from Vestolit) | | | | | | | | | | |
| DINP | 50 | | | | | | | | | |
| (VESTINOL 9, Evonik Industries AG) | | | | | | | | | | |
| Epox. 2-ethylhexyl fatty acid ester | | 50 | | | | | | | | |
| (ex. tall oil; Drapex 4.4 - from Galata) | | | | | | | | | | |
| Epox. isononyl fatty acid ester | | | 50 | | | | | | | |
| (ex. linseed oil; Example 2) | | | | | | | | | | |
| Epox. isononyl fatty acid ester | | | | 50 | | | | | | |
| (ex. tall oil; Example 4a; EN/FA: 1.42) | | | | | | | | | | |
| Epox. isodecyl fatty acid ester | | | | | 50 | | | | | |
| (ex. tall oil; Example 6) | | | | | | | | | | |
| Epox. isononyl fatty acid ester | | | | | | 50 | | | | |
| (ex. tall oil; Example 4b; EN/FA: 1.21) | | | | | | | | | | |
| Epox. isononyl fatty acid ester | | | | | | | 50 | | | |
| (ex. tall oil; Example 4c; EN/FA: 0.74) | | | | | | | | | | |
| Epox. isononyl fatty acid ester | | | | | | | | 50 | | |
| (ex. soya, PLS Green 9; from Petrom) | | | | | | | | | | |
| Epox. isononyl fatty acid ester | | | | | | | | | 50 | |
| (ex. rapeseed, Example 8) | | | | | | | | | | |
| Epox. 2-ethylhexyl fatty acid ester | | | | | | | | | | 50 |
| (ex. soya, PLS Green 8; from Petrom) | | | | | | | | | | |
| Drapex 39 | 3 | 3 | 3 | 3 | 3 | 3 | 3 | 3 | 3 | 3 |
| Mark CZ 149 | 2 | 2 | 2 | 2 | 2 | 2 | 2 | 2 | 2 | 2 |

*Polymer composition comprising an inventive ester
EN/FA: average number of epoxide groups per fatty acid

In the formulations, the products correspond to those from the synthesis procedures from Examples 2, 4a, 4b, 4c, 6 and 8.

In addition to the 50 parts by weight of plasticizer, each formulation also contains 3 parts by weight of an epoxidized soybean oil as co-stabilizer (Drapex 39, from Galata), and also 2 parts by weight of a Ca/Zn-based heat stabilizer (Mark CZ 149, from Galata). The plasticizers were conditioned to 25° C. prior to addition. First the liquid constituents and then those in powder form were weighed out into a PE beaker. The mixture was stirred by hand with a paste spatula until there was no longer any unwetted powder. The mixing beaker was then clamped into the clamping apparatus of a dissolver-stirrer. Before the stirrer was immersed into the mixture, the speed was adjusted to 1800 revolutions per minute. After the stirrer was switched on, stirring took place until the temperature on the digital display of the thermosensor reached 30.0° C. This ensured that homogenization of the plastisol was achieved with a defined energy input. The plastisol was thereafter immediately conditioned at 25.0° C.

3. Measurement of Plastisol Viscosities

The viscosities of the PVC plastisols were measured using a Physica MCR 101 (from Anton-Paar), using the rotation mode and the “CC27” measurement system.

The plastisol was initially homogenized once more in the mixing vessel by stirring with a spatula, then introduced into the measurement system and subjected to isothermal measurement at 25° C. The following points were targeted during the measurement:

1. a pre-shear of 100 s⁻¹ for a period of 60 s, during which no measurement values were recorded (to even out any thixotropic effects).

2. a downward shear-rate progression, starting at 200 s⁻¹ and ending at 0.1 s⁻¹, divided into a logarithmic series of 30 steps each of 5 seconds' measurement point duration.

As a rule (unless otherwise specified), the measurements were carried out after storage/maturation of the plastisols for 24 hours. Between the measurements, the plastisols were stored at 25° C.

Table 3 below shows the viscosities for each of the PVC pastes at a shear rate of 100 s⁻¹. The paste number here correlates with the formulation number from Table 2.

TABLE 3

| Paste No. | 1 | 2 | 3* | 4* | 5 | 6* | 7 | 8 | 9 | 10 |
|---|---|---|---|---|---|---|---|---|---|---|
| Paste viscosity after 24 h | 4.0 | 1.8 | 2.6 | 1.9 | 1.8 | 2.1 | 1.0 | 2.5 | 2.1 | 2.2 |
| (100 s⁻¹) in Pas | | | | | | | | | | |

*Pastes comprising an inventive ester

In comparison with the industry standard DINP (1), all of the pastes based on epoxidized fatty acid esters (2 to 10) show distinctly reduced viscosities. Low paste viscosities imply more effective and more rapid processing of the PVC pastes, and are therefore desirable.

4. Gelling Behaviour

The gelling behaviour of the pastes was studied in a Physica MCR 101 in oscillation mode using a plate/plate measurement system (PP25), which was operated with shear-stress control. An additional temperature-regulating hood was attached to the equipment in order to homogenize heat distribution and achieve a uniform sample temperature. The settings for the parameters were as follows:

Mode: temperature gradient
starting temperature: 25° C.
final temperature: 180° C.
heating/cooling rate: 5° C./min
oscillation frequency: 4-0.1 Hz ramp logarithmic
angular frequency omega: 10 1/s
number of measurement points: 63
measurement point duration: 0.5 min
automatic gap adjustment F: 0 N
constant measurement point duration
gap width 0.5 mm

Measurement Procedure:

A spatula was used to apply a drop of the plastic material to be measured, free from air bubbles, to the lower plate of the measurement system. Care was taken here to ensure that some paste could exude uniformly out of the measurement system (not more than about 6 mm overall) after the measurement system had been closed. The temperature-regulating hood was then positioned over the specimen, and the measurement was started. The so-called complex viscosity of the paste was determined as a function of the temperature. Since a certain temperature is attained within a time span (determined by the heating rate of 5° C./min.), information is obtained about the gelling rate of the measured system, as well as about its gelling temperature. The onset of the gelling process was discernible in a sudden marked rise in the complex viscosity. The earlier the onset of this viscosity rise, the better the gellability of the system.

The measurement curves obtained were used to determine the cross-over temperature. This method computes the point of intersection for the two y-variables chosen. It is used to find the end of the linear viscoelastic region in an amplitude sweep (y: G′, G″; x: gamma), in order to find the crossing frequency in a frequency sweep (y: G′, G″; x: frequency) or in order to ascertain the gel time or cure temperature (y: G′, G″; x: time or temperature). The cross-over temperature documented here corresponds to the temperature of the first intersection of G′ and G.

The results are shown in Table 4. The paste number here correlates with the formulation number from Table 2.

TABLE 4

| Paste No. | 1 | 2 | 3* | 4* | 5 | 6* | 7 | 8 | 9 | 10 |
|---|---|---|---|---|---|---|---|---|---|---|
| Cross-over temperature ° C. | 75.9 | 72.2 | 70.2 | 72.2 | 79.5 | 73.8 | 81.5 | 77.8 | 80.4 | 73.0 |

*Pastes comprising an inventive ester

In comparison to the pastes comprising DINP (1) and the isodecyl ester (5), the inventive pastes (3), (4) and (6) exhibit a significantly lower crossover temperature. This is synonymous with accelerated gelling. Pastes with epoxidized isononyl fatty acid esters which have an average number of epoxide groups per fatty acid of less than 1, or whose fatty acids originate from other oils (7, 8, 9), possess a much higher cross-over temperature.

For further investigations on plasticized PVC specimens, gelled 1 mm polymer films were produced from the corresponding plastisols (gelling conditions in the Mathis oven: 200° C./2 min.).

5. Thermal Stabilities

The thermal stability measurements were carried out on a Thermotester (model LTE-TS from Mathis AG). The sample frame for the thermal stability measurement is fitted with 14 aluminium rails. The aluminium rails serve as sample holders, in which samples up to a maximum width of 2 cm are placed. The sample length is 40 cm.

The edges of the foils under investigation were removed using a guillotine, and the foils were cut to give rectangles (dimensions: 20 cm×30 cm). Then two strips (20*2 cm) were cut off. The strips were fastened alongside one another into the aluminium rails of the frame for the thermal stability measurement. After establishment of temperature, the frame was slotted into the guide of the Thermotester, and measurement was started. The parameters set on the

Mathis Thermotester were as follows:

Temperature: 200° C.

Interval advance: 28 mm

Interval time: 1 min

Ventilator rotation rate: 1800 rpm

Using a Byk colorimeter (Spectro Guide 45/0 from Byk Gardner), determinations were made of the L* a* b*, including a yellowness index Y in accordance with the D1925 index. To achieve optimum results, the illuminant set was C/2°, and a sample observer was used. The thermal stability strips were then measured on each advance (28 mm). Since the thermal stability strips consist of two 20 cm strips, the measurement was not ascertained at the point of cutting. The measurement values were determined directly on the sample card, behind a white tile. The first measurement value following exceedance of the yellowness index maximum was identified as blackening.

The results are set out in Table 5. The specimen number here correlates with the formulation number from Table 2.

TABLE 5

| Specimen number | 1 | 2 | 3* | 4* | 5 | 6* | 7 | 8 | 9 | 10 |
|---|---|---|---|---|---|---|---|---|---|---|
| Time to blackening (min) | 11 | >14 | >14 | >14 | >14 | >14 | >14 | >14 | >14 | >14 |

The specimens produced from epoxidized fatty acid esters (2 to 10) showed no blackening in the thermotester within the time interval under consideration. The thermal stability is significantly increased as compared with the industry standard DINP (1). This significant increase is a result of the capture by the epoxide function of HCI that has been formed.

6. Plasticizing Effect

The Shore hardness is a measure of the flexibility of a specimen. The greater the extent to which a standardized needle can penetrate the specimen within a defined measurement time, the lower the value of the measurement. The plasticizer with the greatest efficiency produces the lowest Shore hardness value for the same quantity of plasticizer. Since, in the art, formulations/recipes are frequently set to or optimized for a defined Shore hardness, therefore, it is possible with very efficient plasticizers to make a saving of a defined fraction in the formulation, which means a reduction in costs for the processor. For determination of the Shore hardnesses, the pastes produced as described above were poured into circular brass casting moulds with a diameter of 42 mm (initial mass: 20.0 g). The pastes in the moulds were then gelled in a forced air drying cabinet at 200° C. for 30 minutes, removed after cooling, and stored in a conditioning cabinet (25° C.) for at least 24 hours prior to measurement. The thickness of the discs was about 12 mm. The hardness measurements were carried out in accordance with DIN 53 505 using a Zwick-Roell Shore A instrument, with the measurement value being read off after 3 seconds in each case. For each specimen, measurements were carried out at three different locations, and an average was formed.

The results are set out in Table 6. The specimen number here correlates with the formulation number from Table 2.

TABLE 6

| Specimen number | 1 | 2 | 3* | 4* | 5 | 6* | 7 | 8 | 9 | 10 |
|---|---|---|---|---|---|---|---|---|---|---|
| Shore A | 82 | 79 | 79 | 79 | 82 | 79 | 87 | 82 | 83 | 81 |

In comparison to the industry standard DINP, specimen (1), the inventive specimens (3), (4) and (6) exhibit lower Shore hardnesses. The plasticizers of the invention can be used to produce PVC blends which possess better efficiency than when the corresponding DINP is used. As a result, a plasticizer saving can be made, leading to reduced formulation costs. In the case of sample (7), a marked incompatibility on the part of the plasticizer leads to exudation from the specimen. Slight exudation is also exhibited by samples (8), (9) and (10). The result of this is a lower plasticizer fraction in the polymer and, in association with this, an increased Shore hardness. In all relevant applications, the exudation of the plasticizer is intolerable.

The experiments described above have shown that the esters of the invention display good to very good plasticizer properties.

1. An isononyl ester or an isononyl ester mixture of an epoxidized fatty acid or of an epoxidized fatty acid mixture,
wherein:
the fatty acid or the fatty acid mixture is obtained from tall oil or linseed oil, and
the ester or the ester mixture has an average number of epoxide groups per fatty acid of greater than 1.00.

2. The isononyl ester or the isononyl ester mixture of claim 1, wherein the fatty acid or the fatty acid mixture is obtained from tall oil.

3. The isononyl ester or the isononyl ester mixture of claim 1, wherein the fatty acid or the fatty acid mixture is obtained from being linseed oil.

4. The isononyl ester or the isononyl ester mixture of claim 1, having an average number of epoxide groups per fatty acid of being greater than 1.20.

5. The isononyl ester mixture of claim 1, having a fraction of saturated fatty acids of less than 12 area %.

6. The isononyl ester mixture of claim 1, having a fraction of saturated fatty acids of greater than 1 area %.

7. A process for preparing the isononyl ester or the isononyl ester mixture of claim 1, comprising:
recovering the fatty acid or the fatty acid mixture from the tall oil or the linseed oil,
epoxidizing the fatty acid or the fatty acid mixture, and
esterifying the fatty acid or the fatty acid mixture with isononanol.

8. A process for preparing the isononyl ester or the isononyl ester mixture of claim 1, comprising:
recovering a fatty acid ester or a fatty acid ester mixture from the tall oil or the linseed oil,
epoxidizing the fatty acid ester or the fatty acid ester mixture, and
transesterifying the fatty acid ester or the fatty acid ester mixture with isononanol.

9. A method for producing a polymer, comprising:
plasticizing the polymer with the isononyl ester or the isononyl ester mixture of.

10. A method for producing a polyvinyl chloride polymer, comprising:
plasticizing the polyvinyl chloride polymer with the isononyl ester or uthe isononyl ester mixture of.
